(19) United States
(12) Patent Application Publication
(10) Pub. No.: US 2007/0185519 A1
(43) Pub. Date: Aug. 9, 2007

(54) Articulating surgical instrument

(76) Inventors: William L. Hassler JR., Cincinnati, OH (US); Christopher W. Widenhouse, Loveland, OH (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/352,471

(22) Filed: Feb. 7, 2006

(51) Int. Cl. A61B 17/00

(52) U.S. Cl. 606/190

(57) ABSTRACT

A method for aiding a surgical procedure, the method uses the steps of providing a surgical instrument with an elongated shaft having a distal end, a proximal end and a longitudinal axis therebetween, the distal end having an end effector attached thereto, the shaft having a first strip running along the longitudinal axis and attached to the end effector, and a second strip, parallel to the first strip, running along the longitudinal axis from the distal end of the shaft to the distal end of the end effector where it is attached thereto, the end effector comprising a plurality of spaced apart links disposed along the longitudinal axis between the distal end of the end effector the distal end of the shaft such that the strips run through the links, and each link is attached to at least one strip. The method also includes the steps of inserting the instrument to a desired location in a body and curving the instrument by moving the first strip proximally. The method also includes the steps of preventing the first strip from moving proximally beyond a predetermined amount and thereby causing the end effector to bend only up to a predetermined angle, and preventing the links from making contact with one another.

### FIELD OF THE INVENTION

The present invention has application in conventional endoscopic and open surgical instrumentation as well as application in robotic-assisted surgery. The present invention has even further relation to adjustable surgically implantable bands, such as gastric bands for the treatment of obesity.

### BACKGROUND OF THE INVENTION

The percentage of the world's population suffering from morbid obesity is steadily increasing. Severely obese persons are susceptible to increased risk of heart disease, stroke, diabetes, pulmonary disease, and accidents. Because of the effect of morbid obesity to the life of the patient, methods of treating morbid obesity are being researched.

Numerous non-operative therapies for morbid obesity have been tried with virtually no permanent success. Dietary counseling, behavior modification, wiring a patient's jaws shut, and pharmacological methods have all been tried, and failed to correct the condition. Mechanical apparatuses for insertion into the body through non-surgical means, such as the use of gastric balloons to fill the stomach have also been employed in the treatment of the condition. Such devices cannot be employed over a long term, however, as they often cause severe irritation, necessitating their periodic removal and hence interruption of treatment. Thus, the medical community has evolved surgical approaches for treatment of morbid obesity.

Most surgical procedures for treatment of morbid obesity may generally be classified as either being directed toward the prevention of absorption of food (malabsorption), or restriction of stomach to make the patient feel full (gastric restriction) The most common malabsorption and gastric restriction technique is the gastric bypass. In variations of this technique, the stomach is horizontally divided into two isolated pouches, with the upper pouch having a small food capacity. The upper pouch is connected to the small intestine, or jejunum, through a small stoma, which restricts the processing of food by the greatly reduced useable stomach. Since food bypass much of the intestines, the amount of absorption of food is greatly reduced.

There are many disadvantages to the above procedure. Typically the above mentioned procedure is performed in an open surgical environment. Current minimally invasive techniques are difficult for surgeons to master, and have many additional drawbacks. Also, there is a high level of patient uneasiness with the idea of such a drastic procedure which is not easily reversible. In addition, all malabsorption techniques carry ongoing risks and side effects to the patient, including malnutrition and dumping syndrome.

Consequently, many patients and physicians prefer to undergo a gastric restriction procedure for the treatment of morbid obesity. One of the most common procedures involves the implantation of an adjustable gastric band. Examples of an adjustable gastric band can be found in U.S. Pat. No. 4,592,339 issued to Kuzmak; RE 36176 issued to Kuzmak; U.S. Pat. No. 5,226,429 issued to Kuzmak; U.S. Pat. No. 6,102,922 issued to Jacobson and U.S. Pat. No. 5,601,604 issued to Vincent, all of which are hereby incorporated herein by reference. In accordance with current practice, a gastric band is operatively placed to encircle the stomach. This divides the stomach into two parts with a stoma in-between. An upper portion, or a pouch, which is relatively small, and a lower portion which is relatively large. The small partitioned portion of the stomach effectively becomes the patients new stomach, requiring very little food to make the patient feel full.

Once positioned around the stomach, the ends of the gastric band are fastened to one another and the band is held securely in place by folding a portion of the gastric wall over the band and closing the folded tissue with sutures placed therethrough thereby preventing the band from slipping and the encircled stoma from expanding.

However, positioning the band around the stomach is often difficult. The band needs to be placed around the stomach, including the posterior side which the physician has little access to. One commercially available product available for the physician to do this is the Goldfinger® sold by Ethicon Endo-Surgery, Inc., Cincinnati Ohio. The instrument basically comprises a straight shaft that has an end which starts out straight but can progressively curve by actuation of a device at the instruments proximal end. Similar devices are also shown in patent literature such as the device disclosed in U.S. Pat. No. 5,467,763 issued to McMahon et al. on Nov. 21, 1995 which is hereby incorporated herein by reference.

When using such a device, the physician uses it first as a blunt dissector to make what is referred to as the retrogastric tunnel. This is a tunnel behind the stomach which goes through the connective tissue surrounding the stomach just below the gastro-esophageal junction. Thereafter, the band is connected to the distal end of the device with suture, and the above described device drives or pulls the band through the tunnel. A straight instrument would have difficulty doing this, but an articulating one which can curve can do the job nicely.

However, there has been a desire to improve upon the commercially available articulating surgical devices.

### SUMMARY OF THE INVENTION

In accordance with the present invention there is provided a method for aiding a surgical procedure, the method uses the steps of providing a surgical instrument with an elongated shaft having a distal end, a proximal end and a longitudinal axis therebetween, the distal end having an end effector attached thereto, the shaft having a first strip running along the longitudinal axis and attached to the end effector, and a second strip, parallel to the first strip, running along the longitudinal axis from the distal end of the shaft to the distal end of the end effector where it is attached thereto, the end effector comprising a plurality of spaced apart links disposed along the longitudinal axis between the distal end of the end effector the distal end of the shaft such that the strips run through the links, and each link is attached to at least one strip. The method also includes the steps of inserting the instrument to a desired location in a body and curving the instrument by moving the first strip proximally. The method also includes the steps of preventing the first strip from moving proximally beyond a predetermined amount and thereby causing the end effector to bend only up to a predetermined angle, and preventing the links from making contact with one another.

### DETAILED DESCRIPTION OF THE DRAWINGS

The novel features of the invention are set forth with particularity in the appended claims. The invention itself, however, both as to organization and methods of operation, together with further objects and advantages thereof, may best be understood by reference to the following description, taken in conjunction with the accompanying drawings in which:

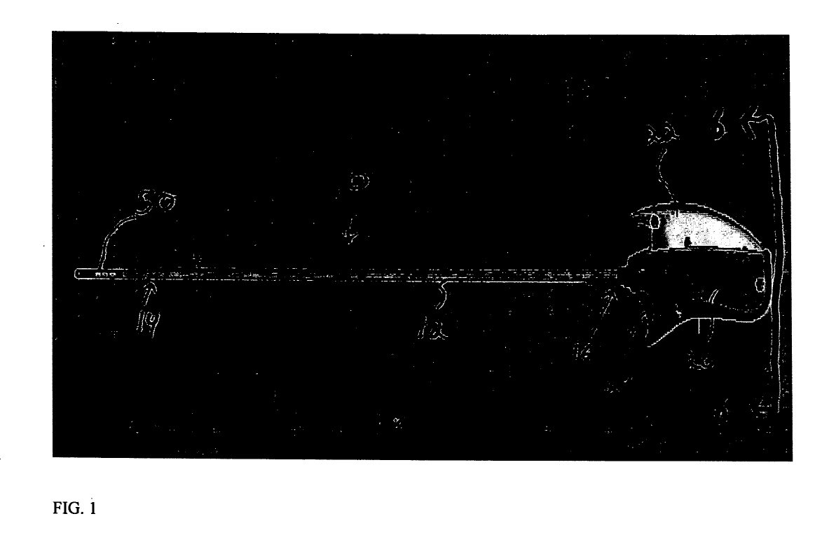

FIG. 1 is a plan view of device 10 made in accordance with the present invention.

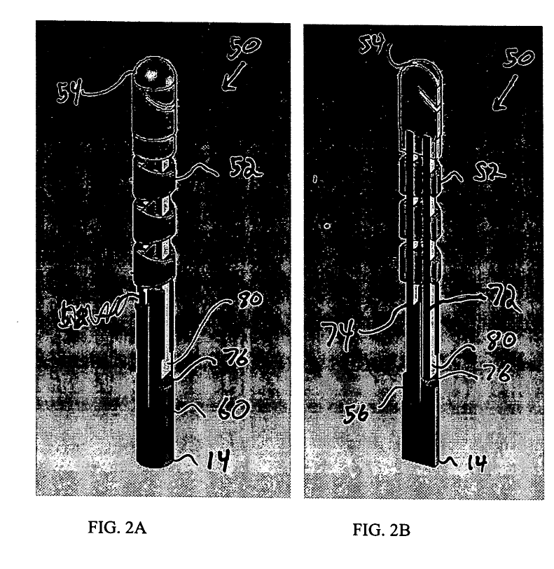

FIG. 2A is a perspective view an end effector for device 10 made in accordance with the present invention.

FIG. 2B is a cross-section view of FIG. 1, taken along lines 2B-2B.

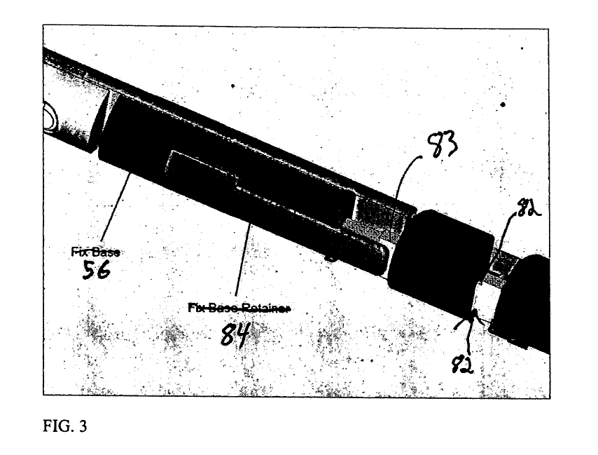

FIG. 3 is a close up view showing a portion of the end effector shown in FIG. 2B

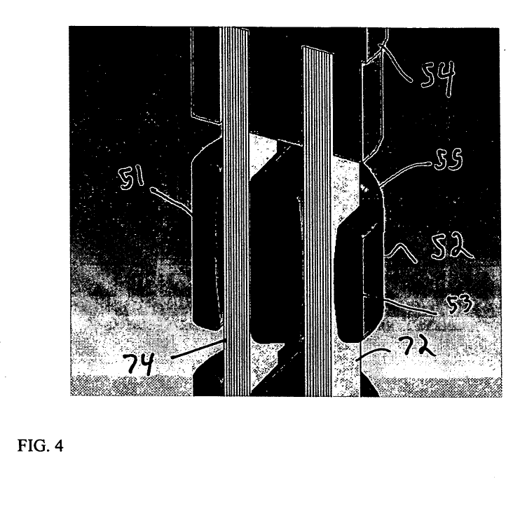

FIG. 4 is another close up view showing a portion of the end effector shown in FIG. 2B.

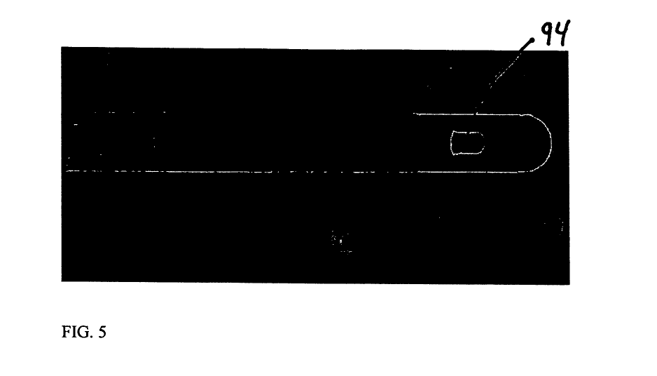

FIG. 5 is a plan view of the end effector shown in FIG. 2A.

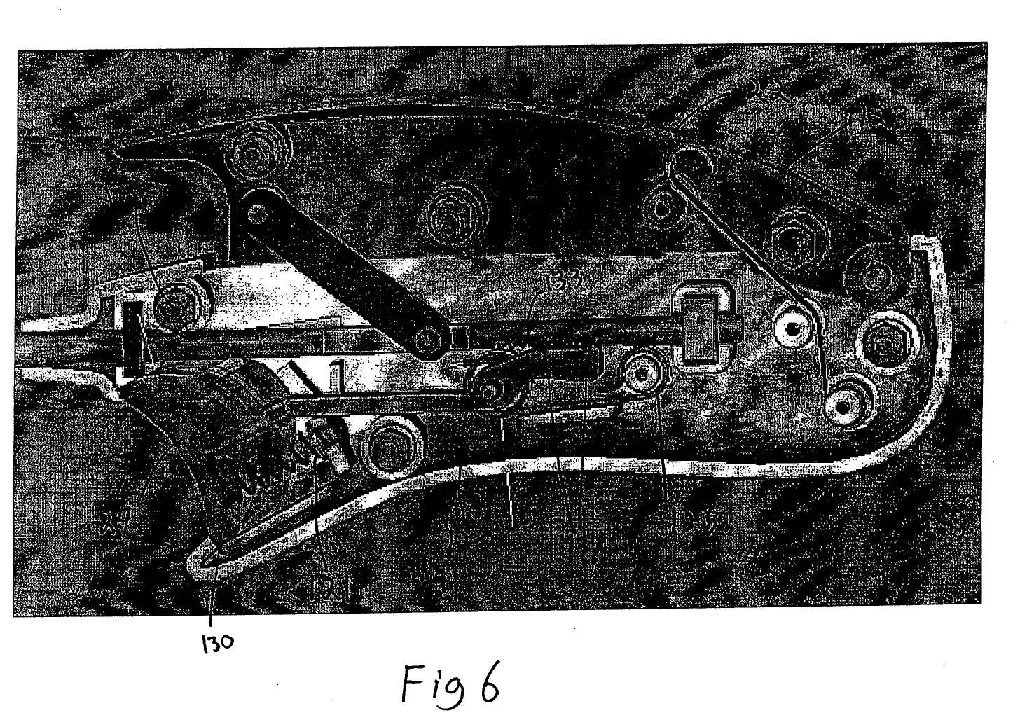

FIG. 6 is a cross-section view of the handle shown in FIG. 1, taken along lines 6-6.

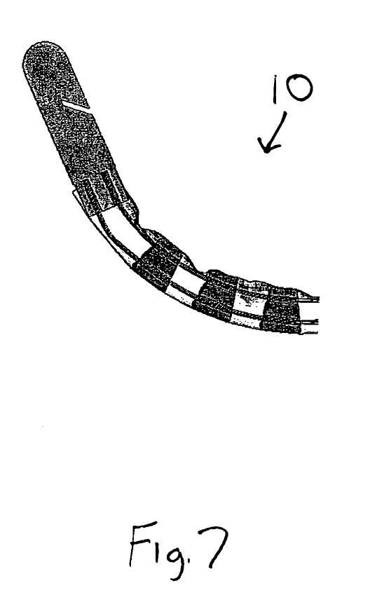

FIG. 7 is a plan view of the end effector shown in its articulated state.

### DETAILED DESCRIPTION OF THE INVENTION

Referring now to the drawings wherein like numerals indicate the same element throughout the views, there is shown in FIG. 1 a device 10, for aiding in a surgical procedure. Device 10 has an elongated shaft 12 with a distal end 14, a proximal end 16 and a longitudinal axis 18 therebetween. The proximal end 16 has a handle 20 attached thereto, and the distal end 14 has an end effector 50 attached thereto. Handle 20 has an actuator 22, and an actuator release 24 attached thereto. As will be apparent later, the handle is held in the surgeon's hand with the thumb portion of the surgeon's palm resting over actuator 22. The angle that end effector 50 bends is controlled by the amount that the actuator is depressed. The more actuator 22 is depressed into the handle, the larger the angle of end effector articulation. However, before actuator 22 can be moved, the surgeon must release it by pressing the actuator release 24 into the instrument handle, and holding it there while the articulation angle is changed. In addition, to release the end effector articulation causing it to go straight, the release trigger must be depressed into the handle.

As seen from FIGS. 2A, 2B, and 3, the end effector includes a plurality of spaced apart links 52 disposed along the longitudinal axis between the distal end of the end effector 54 the distal end of the shaft 14. Shaft 12 has a first strip 72 running along the longitudinal axis and through links 52 from the handle 20 to a distal end of the end effector 54 where it is attached thereto. Device 10 also includes a second strip 74 running along the longitudinal axis through links 52 from the distal end 14 of shaft 12. where it is rigidly attached thereto, to distal end 54 of end effector 50. First and Second Strips are preferably made from stainless steel or any other suitable material known to those skilled in the art. In addition, strips 72 and 74 and can actually comprise a bundle or plurality of strips. Having a plurality of juxtapose strips provide for greater stiffness while still providing good bending. The Fixed base 56 holds the second strip or bundle of strips 74. Base 56 and strips 74 are is rigidly attached to the shaft 12. The Sliding base 60, which pulls on the first strip or plurality of strips 74 in a proximal direction, causes end effector 50 to bend or articulate in a direction towards first strip 72.

Both sets of strips 72 and 74 are attached to the distal end of end effector 54. Since the individual strips act as leaves in a leaf spring, they have to move in a linear direction with respect to each other. Because the leaves are rigidly fixed in the tip, they therefore have to slide with respect to each other within the pockets 76 of the bases 56 and 60 that hold them during actuation of the articulating tip. The larger the angle achieved during tip articulation, the more these strips will slide with respect to each other within the base pockets 76. These pockets have to be significantly longer than the length of the square tabs 80 located at the bottom of the strips being held. This provides adequate clearance needed for the full range of sliding motion of the strips with respect to each other.

Links 52 act as anti-buckling devices for the strips 72 and 74, which hold the two sets or bundles of strips 72 and 74 at a fixed distance from each other, and keep the sets of strips, or leaf springs from buckling. These links also keep the individual strips or leaves from buckling with respect to each other within the bundle so that they act more like a single beam in bending, reinforcing each other like a normal leaf spring. These links are made in two identical halves that will eventually be designed to be pressed fit into each other to hold the strips together, and form a single link.

Strips 72 in the movable base 60 can be covered by a thin Teflon, or other lubricious material that keeps them from popping out of the pocket 76, and helps reduce the friction of the base sliding against the inside of the instrument shaft. As seen from the figures, strip or strips 72 have a series of notches 82 to hold links 52 in place. Strip or strips 74 can be held in place by a stainless steel member or fixed base retainer 84 that extends beyond the end of the fixed base 56. Retainer 84 also retains the notched strips 74 in the pocket, and helps improve the stiffness of the tip.

As seen in FIG. 4, links 52 have an the internal concave contour 51 curved in the direction of articulation (concaved). The radius of curvature of contour feature 51 is less than the radius of curvature of strip 74 when it is bent to its maximum angle to help prevent binding. If the internal slots of the links were not curved in this fashion, then the strips would be forced into bending only between the links, and this would probably result in plastic deformation of the strips, at low force levels. Also, please note the radiused corners where the internal contour cavities intersect with both the top and bottom face of the link. This radius avoids constraining the strips during articulation.

The slots 55 in links 52 containing strips 72 has a contour 53 curvature (convexed). This curvature is to keep the link from pinching the strips 72 when they are being pulled through the link 52. The friction between the strips 72 and the link 52 during articulation will tend to cause the link to rotate slightly downwards which would tend to bind the links 52 and strips 72. By having this reverse curvature, this frictional binding is minimized, and its' effect on the force to actuate the articulating tip is also minimized. The larger this binding friction, the less round the articulation will be. Put another way, The higher the friction, the more bending will occur at the base of the tip, and less at the top. This might eventually require the use of a lubricant in the links such as Sodium Sterate.

As seen from FIG. 5, end effector 54 can be covered by a cylindrical sleeve, or cover 90 to prevent human tissue from catching on the links, and strips, resulting in tissue trauma. Cover 90 can be made from an elstomer such as silicone or a polyurethane thermoplastic elastomer, such as Pellethane (heat shrink tubing). End effector 50 acts as a blunt dissector when doing a surgical procedure such as those described in the background section herein. The dissection tip also has two different notch features. There is an angled notch 92 which can receive a suture loop on the end of an adjustable gastric band. There is also a keyhole feature 94 which can receive a band pulling tab present on many adjustable gastric bands.

The links 52 should also be as long as possible but also cannot be allowed to touch each other as the tip articulates. Causing them to touch may upset the shape the tip will take, as well as the uniformity of the force loading in the tip. It is also necessary that the links not touch each other even under load, as these links are not designed to take axial loads, only the anti-buckling loads that are small.

Referring now to FIG. 6, there is shown the handle 20 of device 10. Handle 20 is very similar to the handle of the device described in U.S. patent application Ser. No. 10/741875, publication no. US-2004-0254537-A1, which is hereby incorporated herein by reference. When the surgeon depresses the articulation release trigger 24 with his index finger, the shaft 130 on the back of the release trigger 24 pushes on the ratchet pall arm 120 causing it to rotate. This rotation causes the teeth 132 on the other side of arm 120 to disengage from the matching ratchet teeth 133 on the articulation drive shaft 122 which is connected to the sliding base 60 pulling it and strips 72 proximally. This allows drive shaft 122 to move along the axis of the instrument and accomplish articulation of the tip.

While the ratchet is disengaged by depressing the release trigger, the surgeon can then either depress the actuator 22 to articulate the tip or release the lever allowing the actuator reset spring 121 to push the actuator back up, and thereby de-articulate the end effector. When the surgeon sees that the end effector is at the desired articulation angle, he can then release the actuator 22, which will move back forward due to the force supplied by the release trigger reset spring 123. Correspondingly, pall arm reset spring 125 will then cause the ratchet pall arm 120 to rotate back so that the ratchet teeth 132 will then re-engage the teeth 133 on the articulation drive shaft 122, thereby preventing any change in the setting of the articulation mechanism. This ratchet arrangement is designed so as to minimize the risk of any unintended changes in the articulation of the instrument tip. This is accomplished by having the surgeon holding the release trigger 24 in the depressed position while changing the tip articulation angle. The instrument articulation cannot be readily changed without the depression of this trigger.

The actuator also includes a means for preventing the first strip from moving proximally beyond a predetermined amount and thereby causing the end effector to bend only up to a predetermined angle. This is done by limiting the movement of the drive shaft 122, by correctly sizing the length of the teeth 133. A preferred maximum angle for end effector articulation is 90 degrees. In addition, the predetermined angle is such that the links never make contact with one another. FIG. 7 shows the instrument in its articulated position.

Lastly, it is preferred that device 10 be sterilized. This can be done by any number of ways known to those skilled in the art including beta or gamma radiation, ethylene oxide, steam.

It will become readily apparent to those skilled in the art that the above invention has equally applicability to other types of implantable bands. For example, bands are used for the treatment of fecal incontinence. One such band is described in U.S. Pat. No. 6,461,292 which is hereby incorporated herein by reference. Bands can also be used to treat urinary incontinence. One such band is described in U.S. Patent Application 2003/0105385 which is hereby incorporated herein by reference. Bands can also be used to treat heartburn and/or acid reflux. One such band is described in U.S. Pat. No. 6,470,892 which is hereby incorporated herein by reference. Bands can also be used to treat impotence. One such band is described in U.S. Patent Application 2003/0114729 which is hereby incorporated herein by reference.

While preferred embodiments of the present invention have been shown and described herein, it will be obvious to those skilled in the art that such embodiments are provided by way of example only. Numerous variations, changes, and substitutions will now occur to those skilled in the art without departing from the invention. For example, as would be apparent to those skilled in the art, the disclosures herein have equal application in robotic-assisted surgery. In addition, it should be understood that every structure described above has a function and such structure can be referred to as a means for performing that function. Accordingly, it is intended that the invention be limited only by the spirit and scope of the appended claims.

What is claimed is:

1. A method for aiding a surgical procedure, said method comprising the steps of:
a. providing a surgical instrument comprising an elongated shaft having a distal end, a proximal end and a longitudinal axis therebetween, said distal end having an end effector attached thereto, said shaft having a first strip running along said longitudinal axis and attached to said end effector, and a second strip, parallel to said first strip, running along said longitudinal axis from said distal end of said shaft to said distal end of said end effector where it is attached thereto, said end effector comprising a plurality of spaced apart links disposed along said longitudinal axis between said distal end of the end effector said distal end of said shaft such that said strips run through said links, and each link is attached to at least one strip;
b. inserting said instrument to a desired location in a body;
c. curving said instrument by moving said first strip proximally;
d. preventing said first strip from moving proximally beyond a predetermined amount and thereby causing said end effector to bend only up to a predetermined angle; and
e. preventing said links from making contact with one another.

2. The method of claim 1 further including the step of sterilizing said device after steps a-e.

3. The method of claim 1 further including the step of dissecting tissue with said end effector.
